SEQUENCE LISTING (2 sequences)

SEQ ID NO: 1 - DNA, 20 bp, Synthetic Sequence
  Description of synthetic sequence Primer
attaatattatgtaaagtga

SEQ ID NO: 2 - DNA, 20 bp, Synthetic Sequence
  Description of synthetic sequence Primer
cttacaaccattcacccaca

(19) United States
(12) Patent Application Publication
(10) Pub. No.: US 2003/0104464 A1
(43) Pub. Date: Jun. 5, 2003

(54) Method for the high-parallel analysis of polymorphisms

(76) Inventors: Kurt Berlin, Stahnsdorf (DE); Ivo Glynne Gut, Paris (FR)

Correspondence Address: KRIEGSMAN & KRIEGSMAN, 665 FRANKLIN STREET, FRAMINGHAM, MA, 01702, US

(21) Appl. No.: 10/311,813

(22) PCT Filed: Jun. 19, 2001
(86) PCT No.: PCT/DE01/02273

(30) Foreign Application Priority Data
Jun. 19, 2000 (DE) 10029914.8

(51) Int. Cl. C12Q 1/68; C12M 1/34

(52) U.S. Cl. 435/6; 435/287.2

(57) ABSTRACT

Disclosed is a method for the high-parallel characterisation of polymorphisms, especially SNPs. Said method can also be used for the simultaneous or separate detection of DNA methylation. First, a set of probes provided with at least one characteristic detectable identifying mark for a respective probe is connected to an addressed surface. A nucleic acid to be examined is then hybridised to these probes and the probes are extended in an allele-specific enzymatic reaction. The type and the occurrence of said allele-specific reaction determine whether the respective probe is enzymatically decomposed. Finally, the remaining allele-specific products are analysed and the existing alleles in the extracted nucleic acid samples are determined.

The present invention describes a method for the highly parallel analysis of polymorphisms, particularly SNPs. The method can be used simultaneously or in a separate experiment for the analysis of DNA methylation.

The Human Genome Project, the first sequencing of the human genome, will be completed in the next few years. Due to this Project, it will be possible to identify all approximately 100,000 genes. The sequence information opens up unexpected possibilities for the clarification of gene functions. This in turn can become a driving force in pharmacogenetics and pharmacogenomics. Pharmacogenetics and pharmacogenomics relate to the application of medications as a function of genotype. The effectiveness of medications will be increased in this way. The necessary intermediate step is the determination of polymorphisms and genotypes which are associated with a specific response. Thus, continuously more efficient genotyping methods will be required.

Currently there are two categories of polymorphic markers, which are utilized for genotyping: microsatellites and single nucleotide polymorphisms (SNPs). Microsatellites are highly polymorphic, i.e., they have a multiple number of alleles. They are characterized in that a repetitive sequence element, with a different number of repetitions for different alleles, is flanked by conserved sequences. On average, there is one microsatellite marker per 1 million bases. A map of 5,000 positioned microsatellite markers was published by CEPH (Dil, C. et al. Nature, Mar. 14, 1994). Microsatellites are genotyped by determining the size of PCR products using primers of the conserved, flanking sequence. The fluorescently labeled PCR products are separated on gels.

There are comparatively few SNP markers that have been described. A map with 300,000 SNP markers is currently being developed by the SNP Consortium and will be made accessible in the public domain. There is a handful of genotyping methods for SNPs. Several are based on the separation of products on gels, such as the oligonucleotide ligase assay (OLA). The latter is particularly suitable for an intermediate throughput. Others rely on pure hybridization, however, which does not have the same stringency. DNA arrays (DNA chips) are suitable for the analysis of a large number of SNPs in a limited number of individuals. Up to now, examples have been shown in which 1,500 SNPs were genotyped on one DNA chip. The real strength of DNA chips lies in approaches such as resequencing und expression analysis. Approaches which apply primer extension have been presented. If one is working with fluorescently labeled terminator bases, these approaches have the advantage that the results can be compiled with a simple ELISA reading device.

There are several SNP genotyping methods, which use mass spectrometry for analysis. These have the basic advantage that the allele-specific products are a physical representation of the products and not a fluorescent signal that must be assigned indirectly to the product.

One method that was recently presented is the Invader assay, and also the Invader Squared assay as a variant thereof (T. Griffin and L. M. Smith Proceedings of the ASMS 1998). At least two oligonucleotides, which cover a known SNP, are used for this method. One oligonucleotide covers the sequence of the 5′ side directly up to the SNP, so that the SNP is connected to the 3′ end of this oligonucleotide. For the most part, two other oligonucleotides, each of which covers one allele of the polymorphism and has a different 5′ overhang, are hybridized to the system. A structurally reactive endonuclease removes the 5′ overhang from the completely complementary oligonucleotide. The decapitated overhang is analyzed by means of mass spectrometry and is used for the identification of the allele. A disadvantage of the described method is that the products must be basically purified prior to the mass-spectrometric analysis. Magnetic beads, which are not simple to handle, are used for this purification. This is a basic disadvantage of many genotyping methods which use mass spectrometry for the analysis.

Another genotyping method is the Taq Man Assay. In this method, a fluorescence extinguisher is separated enzymatically, in an allele-specific manner, by an oligonucleotide bearing a fluorescent dye.

Matrix-assisted laser desorption/ionization time-of flight mass spectrometry (MALDI) has revolutionized the analysis of biomolecules (Karas, M. & Hillenkamp, F. Anal. Chem. 60, 2299-2301 (1988)). MALDI has been applied in different variants to the analysis of DNA. The variants extend from primer extension to sequencing (Liu, Y.-H., et al. Rapid Commun. Mass Spectrom. 9, 735-743 (1995); Ch'ang, L.-Y., et al. Rapid Commun. Mass Spectrom. 9, 772-774 (1995); Little, D. P., et al. J. Mol. Med. 75, 745-750 (1997); Haff, L. & Smirnov, I. P. Genome Res. 7, 378-388 (1997), Fei, Z., Ono, T. & Smith, L. M. Nucleic Acids Res. 26, 2827-2828 (1998); Ross, P., Hall, L., Smirnov, I. & Haff, L. Nature Biotech. 16, 1347-1351 (1998); Ross, P. L., Lee, K. & Belgrader, P. Anal. Chem. 69, 4197-4202 (1997); Griffin, T. J., Tang, W. & Smith, L. M. Nature Biotech. 15, 1368-1372 (1997)). The greatest disadvantage of these methods is that all require a basic purification of the products prior to the MALDI analysis. Spin column purification or the use of magnetic bead technology or reversed-phase purification are necessary.

The analysis of DNA in MALDI is very dependent on the charge state of the product. A 100-fold improvement of the sensitivity in MALDI analysis can be achieved by controlling the charge state of the product to be analyzed, so that only a slight positive or negative excess charge is present. The products modified in this way are also essentially less susceptible to the formation of adducts (e.g. with Na and K, Gut, I. G. and Beck, S. (1995) Nucleic Acids Res., 23, 1367-1373; Gut, I. G., Jeffery, W. A., Pappin, D. J. C. and Beck, S. Rapid Commun. Mass Spectrom., 11, 43-50 (1997)). An SNP genotyping method, which makes use of these conditions, with the name “GOOD Assay” has been proposed recently (Sauer, S. et al., Nucleic Acids Research, Methods online, 2000, 28, e13). A disadvantage is that the overall method permits only a limited degree of multiplexing, and the sample preparation always requires the use of the most modern and expensive pipetting technology.

5-Methylcytosine is the most frequent covalently modified base in the DNA of eukaryotic cells. For example, it plays a role in the regulation of transcription, genetic imprinting and in tumorigenesis. The identification of 5-methylcytosine as a component of genetic information is thus of considerable interest. 5-Methylcytosine positions, however, cannot be identified by sequencing, since 5-methylcytosine has the same base-pairing behavior as cytosine. In addition, in the case of a PCR amplification, the epigenetic information which is borne by the 5-methylcytosines is completely lost.

A relatively new method that has become the most widely used method for investigating DNA for 5-methylcytosine is based on the specific reaction of bisulfite with cytosine, which, after subsequent alkaline hydrolysis, is then converted to uracil, which corresponds in its base-pairing behavior to thymidine. In contrast, 5-methylcytosine is not modified under these conditions. Thus, the original DNA is converted so that methylcytosine, which originally cannot be distinguished from cytosine by its hybridization behavior, can now be detected by “standard” molecular biology techniques as the only remaining cytosine, for example, by amplification and hybridization or sequencing. All of these techniques are based on base pairing, which will now be fully utilized. The prior art, which concerns sensitivity, is defined by a method that incorporates the DNA to be investigated in an agarose matrix, so that the diffusion and renaturation of the DNA is prevented (bisulfite reacts only on single-stranded DNA) and all precipitation and purification steps are replaced by rapid dialysis (Olek, A. et al., Nucl. Acids Res. 1996, 24, 5064-5066). Individual cells can be investigated by this method, which illustrates the potential of the method. Of course, up until now, only individual regions of up to approximately 3000 base pairs long have been investigated; a global investigation of cells for thousands of possible methylation analyses is not possible. Of course, this method also cannot reliably analyze very small fragments of small quantities of sample. These are lost despite the protection from diffusion through the matrix.

An overview of other known possibilities for detecting 5-methylcytosines can be derived from the following review article: Rein, T., DePamphilis, M. L., Zorbas, H., Nucleic Acids Res. 1998, 26, 2255.

With just a few exceptions (e.g. Zechnigk, M. et al., Eur. J. Hum. Gen. 1997, 5, 94-98), the bisulfite technique has only been applied in research. However, short, specific segments of a known gene are always amplified after a bisulfite treatment and either completely sequenced (Olek, A. und Walter, J., Nat. Genet. 1997, 17, 275-276) or individual cytosine positions are detected by a “primer extension reaction” (Gonzalgo, M. L. and Jones, P. A., Nucl. Acids Res. 1997, 25, 2529-2531, WO Patent 95-00669) or an enzyme step (Xiong, Z. and Laird, P. W., Nucl. Acids Res. 1997, 25, 2532-2534). Detection by hybridization has also been described (Olek et al., WO 99 28498).

Other publications which are concerned with the application of the bisulfite technique for the detection of methylation in the case of individual genes are: Xiong, Z. and Laird, P. W. (1997), Nucl. Acids Res. 25, 2532; Gonzalgo, M. L. and Jones, P. A. (1997), Nucl. Acids Res. 25, 2529; Grigg, S. and Clark, S. (1994), Bioassays 16, 431; Zeschnik, M. et al. (1997), Human Molecular Genetics 6, 387; Teil, R. et al. (1994), Nucl. Acids Res. 22, 695; Martin, V. et al. (1995), Gene 157, 261; WO 97 46705, WO 95 15373 and WO 45560.

An overview of the state of the art in oligomer array production can be taken also from a special issue of Nature Genetics which appeared in January 1999 (Nature Genetics Supplement, Volume 21, January 1999), the literature cited therein and U.S. Pat. No. 5,994,065 on methods for the production of solid supports for target molecules such as oligonucleotides with reduced nonspecific background signal.

Probes with multiple fluorescent labels are used for scanning an immobilized DNA array. Particularly suitable for fluorescent labeling is the simple introduction of Cy3 und Cy5 dyes at the 5′-OH of the respective probe. The fluorescence of the hybridized probes is detected, for example, by means of a confocal microscope. The dyes Cy3 and Cy5, in addition to many others, can be obtained commercially.

Genomic DNA is obtained from DNA of cells, tissue or other test samples by standard methods. This standard methodology is found in references such as Fritsch and Maniatis, eds., Molecular Cloning: A Laboratory Manual, 1989.

The object of the present invention is to make available a method for the highly parallel analysis of polymorphisms, which overcomes the disadvantages of the prior art.

The subject of the invention is a method for the highly parallel characterization of polymorhpisms, in which the following steps are conducted:

a. a set of probes is bound to an addressed surface,

b. a nucleic acid to be investigated is hybridized to these probes;

c. the probes are extended in an allele-specific reaction, which depends on the sequence of nucleic acids to be investigated that functions as the template;

d. the probes are treated with a nuclease, which decomposes the unextended probes, but not the extended probes;

e. the allele-specific extension products that remain are analyzed.

It is preferred according to the invention that the address of the surface in step a) is the position (in an oligonucleotide array), a color, a fluorescent label, an isotopic label, a chemical label or a radioactive label.

It is further preferred according to the invention that the nucleic acid to be investigated in step b) is genomic DNA, DNA pretreated with a bisulfite solution, cloned DNA, cDNA, RNA, a PCR product or a ligation product.

It is also preferred according to the invention that the probes are converted to specific products corresponding to step c), as a function of the respective sequence of the template hybridized thereon, by means of a polymerase and modified nucleotide building blocks.

It is further preferred that the probes are converted to specific extension products, corresponding to step c), as a function of the respective sequence of the template hybridized thereon, by means of a ligase and a phosphorylated oligonucleotide.

It is further preferred according to the invention that the extension reaction or the type of extension reaction depends on an SNP (Single Nucleotide Polymorphism) in the sample DNA.

It is particularly preferred according to the invention that the nucleic acid to be investigated is a genomic DNA sample pretreated with a bisulfite solution (=hydrogen sulfite, disulfite) and that the extension reaction or the type of extension reaction depends on the methylation state of cytosine bases in the genomic DNA sample.

A method according to the invention is highly preferred in which SNPs and DNA methylation are investigated simultaneously.

It is preferred according to the invention that at least one nucleotide is attached in the extension reaction, which [nucleotide] cannot be cleaved by a 3′-exonuclease or can be cleaved only with considerably reduced efficiency. In several cases, it is particularly preferred that this nucleotide is a methyl phosphonate, a phosphorothioate, a phosphorodithioate, a methyl phosphorothioate, an alkylated phosphorothioate or dithioate or a derivative of these compounds.

It is further preferred according to the invention that a substituent which hinders decomposition by a 3′-exonuclease is attached at the specified nucleotide base either on the nucleobase itself or on the deoxyribose.

It is further preferred that one uses a 3′-exonuclease in step d). It is particularly preferred according to the invention that phosphodiesterase from Crotalus durissus (snake venom phosphodiesterase), Escherichia coli polymerase I, II, or III, T4 DNA polymerase, T7 DNA polymerase (unmodified), phosphodiesterase II type I-SA or calf thymus 54-kDa polypeptide with 3′-exonuclease activity is used.

It is also preferred according to the invention that the extension products are provided with a detectable label for their detection. It is likewise preferred that complementary oligomers are hybridized to the extension products, which are provided with a detectable label for their detection. It is particularly preferred that the complementary oligomers are oligonucleotides, RNA oligomers or PNA oligomers (Peptide Nucleic Acids). It is further highly preferred that the labels are fluorescent labels and/or that the labels are radionuclides and/or that the labels are removable mass labels, which are detected in a mass spectrometer and/or that the extension products or the complementary oligomers are themselves detected via their mass in a mass spectrometer.

However, it is also preferred according to the invention that the allele-specific extension products are analyzed by means of mass spectrometry and/or that fragments of the allele-specific extension products are analyzed by means of mass spectrometry. It is still further particularly preferred also that matrix-assisted laser desorption/ionization mass spectrometry (MALDI) or electrospray ionization mass spectrometry (ESI) is used for analysis.

For this purpose, it is advantageous according to the invention that the probes or the complementary oligomers are present in a form which is particularly well suitable for mass-spectrometric analysis. It is thus preferred that particularaly good suitability for mass-spectrometric analysis is achieved if the allele-specific products are given a net single positive charge or single negative charge.

It is further preferred according to the invention that a plurality of different probes are [present] on an addressed analysis point of the surface.

It is also preferred according to the invention that known polymorphisms in the DNA to be investigated are genotyped and/or unknown polymorphisms in the DNA to be investigated are identified and/or that cytosine methylations are detected and visualized. It is particularly preferred that known methylation patterns are investigated in the sample to be analyzed.

It is particularly preferred in the method according to the invention that genomic DNA is obtained from a DNA sample, whereby sources for DNA include, e.g., cell lines, blood, sputum, stool, urine, cerebrospinal fluid, tissue embedded in paraffin, for example, tissue from eyes, intestine, kidney, brain, heart, prostate, lungs, breast or liver, histological microscope slides and all possible combinations thereof.

Another subject of the present invention is the use of the method according to the invention for the diagnosis and/or prognosis of adverse events for patients or individuals, whereby these adverse events belong to at least one of the following categories: undesired drug interactions; cancer disorders; CNS malfunctions, damage or disease; symptoms of aggression or behavioral disturbances; clinical, psychological and social consequences of brain damage; psychotic disturbances and personality disorders; dementia and/or associated syndromes; cardiovascular disease, malfunction and damage; malfunction, damage or disease of the gastrointestinal tract; malfunction, damage or disease of the respiratory system; lesion, inflammation, infection, immunity and/or convalescence; malfunction, damage or disease of the body as an abnormality in the development process; malfunction, damage or disorder of the skin, the muscles, the connective tissue or the bones; endocrine and metabolic malfunction, damage or disorder; headaches or sexual malfunction.

In addition, another subject of the present invention is the use of the method according to the invention for distinguishing cell types or tissues or for investigating cell differentiation.

Finally, another subject of the present invention is a kit, containing at least one pair of primers for amplification, a set of probes and enzymes and buffer and instructions for conducting the method according to the invention.

A method is made available for the highly parallel genotyping of polymorphsms. This method goes far beyond the efficiency of existing methods, with respect to simplicity of handling, cost, quality and throughput. It is also suitable for the simultaneous detection of cytosine methylation in nucleic acid samples.

The invention thus describes a method for the highly parallel characterization of polymorphisms.

In the first step of the method, a set of probes is bound to an addressed surface.

Preferably, oligonucleotides, modified oligonucleotides, peptide nucleic acids (PNAs), chimeras of these compound classes or other substances are used as probes, which interact with DNA in a sequence-specific manner.

The respective probe is provided with a characteristic detectable label. In a particularly preferred variant of the method, the addressing of the surface is the position in an oligonucleotide array, a color, a fluorescent label, an isotopic label, a chemical label or a radioactive label.

In the second step of the method, the nucleic acid to be investigated, which preferably consists of genomic DNA, cloned DNA, chemically pretreated DNA, cDNA, RNA, PCR products or ligation products, is hybridized to said probes.

Preferably, the nucleic acids to be investigated comprise a DNA sample, whereby sources for DNA include, e.g., cell lines, blood, sputum, stool, urine, cerebrospinal fluid, tissue embedded in paraffin, for example, tissue from eyes, intestine, kidney, brain, heart, prostate, lungs, breast or liver, histological microscope slides and all possible combinations thereof.

In a particularly preferred variant of the method, the DNA is first treated with a bisulfite solution (disulfite, hydrogen sulfite).

In the third method step, the probes are extended in an allele-specific enzymatic reaction, which depends on the sequence of nucleic acids to be investigated that functions as the template;

In a particularly preferred variant of the method, the probes are converted to specific products, as a function of the respective sequence of the template hybridized thereon, by means of a polymerase and nucleotide building blocks.

In another preferred variant of the method, the probes are converted to specific products, as a function of the respective sequence of the template hybridized thereon, by means of a ligase and a 5′-phosphorylated oligonucleotide.

In a particularly preferred variant of the method, methylation patterns are investigated in the pretreated DNA to be analyzed, and the type or the occurrence of the extension reaction or ligase reaction depends on the specific formation of a potentially methylated position in the nucleic acid sample to be investigated. In this case, it is necessary to pretreat the nucleic acid sample with a bisulfite solution, whereby after alkaline hydrolysis, the unmethylated cytosine bases are present as uracil, while the 5-methylcytosine bases, however, remain unchanged.

In another particularly preferred variant of the method, SNPs are investigated in the pretreated DNA to be analyzed, and the type or the occurrence of the extension reaction or ligase reaction depends on the specific formation of an SNP (Single Nucleotide Polymorphism) in the nucleic acid sample to be investigated.

In a particularly preferred variant of the method, cytosine methylation and SNPs of a nucleic acid sample are investigated in one experiment.

Preferrably, a plurality of different probes are found on an addressed analysis point of the surface.

In a particularly preferred embodiment of the method, in the extension reaction, at least one nucleotide or another unit is attached, which can either not be cleaved by a 3′-exonuclease or can be cleaved only with considerably reduced efficiency. In this way, the decomposition of the extension products, which are characteristic each time for a specific formation of an SNP or a methylation position, is prevented.

Preferably, such units are methyl phosphonates, phosphorothioates, phosphorodithioates, methyl phosphorothioates, alkylated phosphorodithioates or phosphorothioates, or derivatives of these compounds.

Preferably, such units are also nucleotide building blocks, which bear substituents that prevent decomposition by a 3′-exonuclease, either on the nucleobase itself or on the deoxyribose.

It is also preferable in the case of the ligase reaction that an oligonucleotide is attached which contains at least one of the above-named chemical units.

In the fourth step of the method, the unextended probes are decomposed. Preferably, the hybridized nucleic acids to be investigated are removed beforehand.

It is also possible to design the method in such a way that specific extension products, which do not correspond to a specific formation of an SNP or a methylation position or are not assigned to these, are also decomposed.

The decomposition is particularly preferably conducted by a 3′-exonuclease, and again particularly preferably by phosphodiesterase from Crotalus durissus (snake venom phosphodiesterase).

The use of Escherichia coli polymerase I, II, and III, T4 DNA polymerase, T7 DNA polymerase (unmodified), phosphodiesterase II type I-SA or calf thymus 54-kDa polypeptide with 3′-exonuclease activity is also preferred.

In the fifth step of the method, the remaining allele-specific products are analyzed.

The extension products are preferably provided with a detectable label.

In another particularly preferred variant of the method, oligomers that are complementary to the extension products are hybridized. The complementary oligomers are particularly preferably provided with a detectable label.

Particularly preferred are complementary oligomers, oligonucleotides, modified oligonucleotides, peptide nucleic acids (PNAs), chimeras of these compound classes or other substances which interact with DNA in a sequence-specific manner.

In a particularly preferred variant of the method, the detectable labels are fluorescent labels.

In a particularly preferred variant of the method, the detectable labels are radionuclides.

In a particularly preferred variant of the method, the detectable labels are removable mass labels, which are detected in a mass spectrometer.

In another particularly preferred variant of the method, the extension products or the complementary oligomers themselves are detected via their mass in a mass spectrometer.

In a particularly preferred variant of the method, the allele-specific extension products are analyzed by means of mass spectrometry. In another particularly preferred variant, fragments of the allele-specific extension products are analyzed by means of mass spectrometry.

Matrix-assisted laser desorption/ionization mass spectrometry (MALDI) or electrospray ionization mass spectrometry (ESI) is particularly preferably used for analysis.

In a particularly preferred embodiment of the method, the probes or the complementary oligomers are in a form that is particularly well suitable for mass-spectrometric analysis. This particularaly good suitability for mass-spectrometric analysis is achieved preferably if the allele-specific products have a net single positive charge or single negative charge.

Preferably, a plurality of different probes are found on an addressed analysis point of the surface.

Particularly preferred, known polymorphisms in the DNA to be investigated are genotyped according to the method of the invention.

Preferably, unknown polymorphisms in the DNA to be investigated are genotyped* according to the method of the invention. *sic; This has been denoted “identified” in other places in the patent—Trans. Note.

Particularly preferred, cytosine methylations are detected and visualized according to the method of the invention.

Particularly preferred, known methylation patterns in the sample to be analyzed are investigated according to the method of the invention.

The subject of the invention is also the use of the above-described method for the diagnosis and/or prognosis of adverse events for patients or individuals, whereby these adverse events belong to at least one of the following categories: undesired drug interactions; cancer disorders; CNS malfunctions, damage or disease; symptoms of aggression or behavioral disturbances; clinical, psychological and social consequences of brain damage; psychotic disturbances and personality disorders; dementia and/or associated syndromes; cardiovascular disease, malfunction and damage; malfunction, damage or disease of the gastrointestinal tract; malfunction, damage or disease of the respiratory system; lesion, inflammation, infection, immunity and/or convalescence; malfunction, damage or disease of the body as an abnormality in the development process; malfunction, damage or disorder of the skin, the muscles, the connective tissue or the bones; endocrine and metabolic malfunction, damage or disorder; headaches or sexual malfunction.

The subject of the invention is also the use of the above-described method for distinguishing cell types or tissues or for investigating cell differentiation.

The subject of the present invention is also a kit, containing a pair of primers for amplification, a set of probes and enzymes and buffer and instructions for conducting the above-described method.

The method will finally be explained by a drawing.

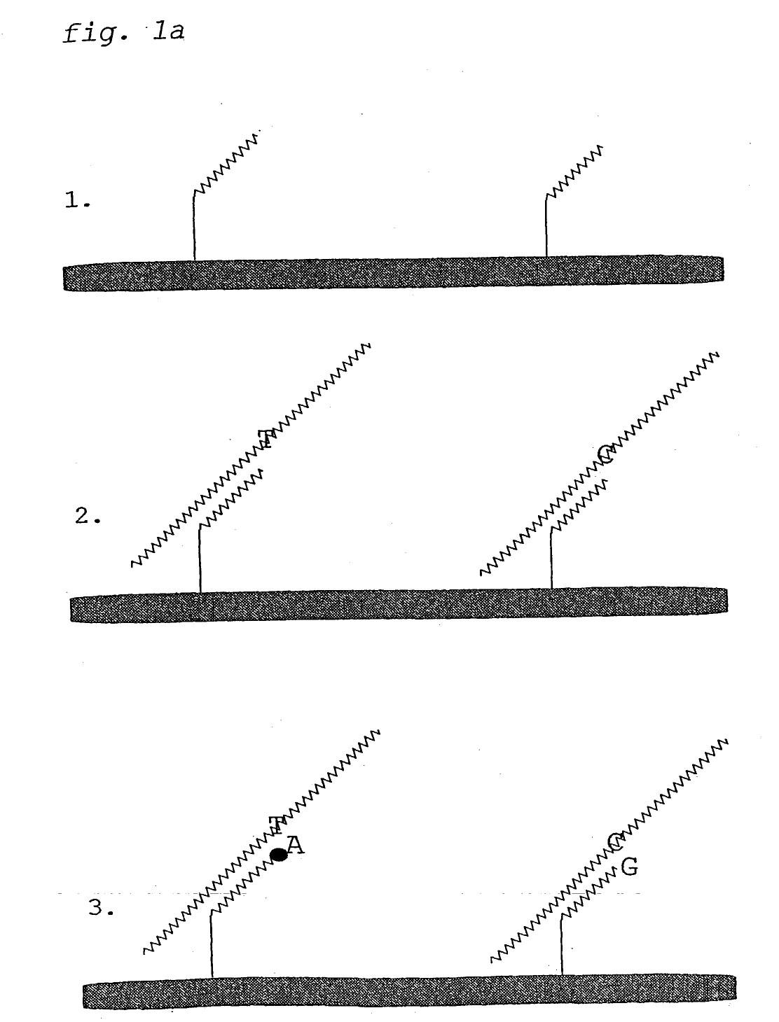

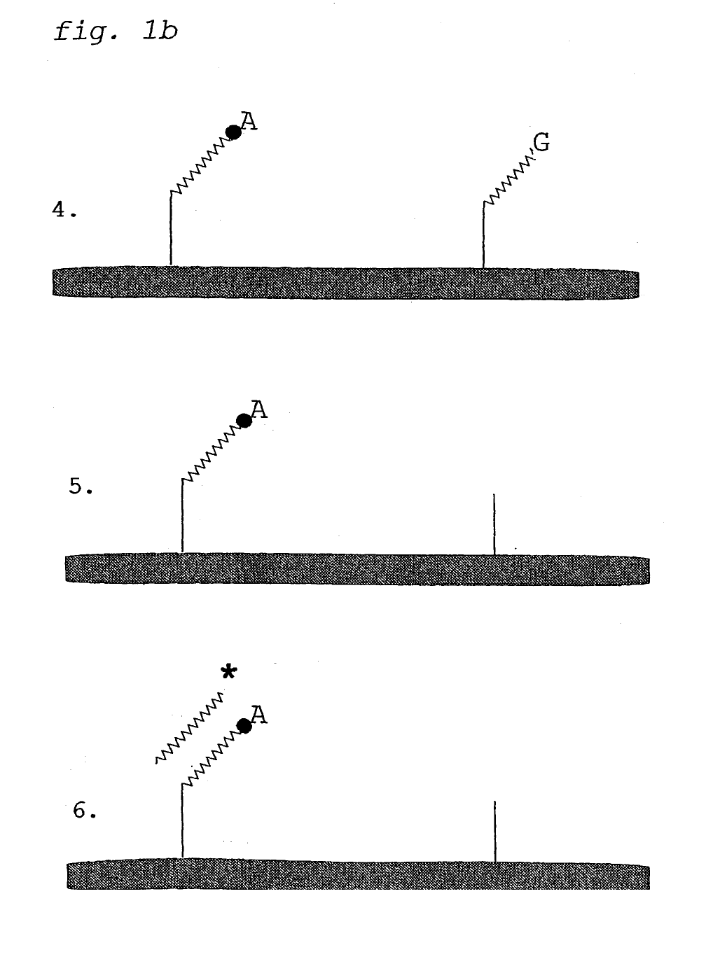

FIG. 1a and 1b illustrate the method steps on an example:

1. First, probes are bound to the addressed surface.

2. Then the nucleic acids to be investigated are hybridized to the probe.

3. Subsequently, the probes are extended in an allele-specific reaction. A unit, for example, a phosphorothioate (a black circle in the figure), which prevents decomposition in the following step, is incorporated with the adenine base only at positions at which a T is present.

4. The nucleic acid to be investigated is removed.

5. Subsequently, the probes, which were not provided with a blocking function in the third step, are enzymatically decomposed.

6. The remaining extension products are analyzed by hybridizing, for example, a complementary oligonucleotide which bears a fluorescent label (characterized by * in the figure) to these remaining extension products.

The following examples explain the invention:

### EXAMPLE 1

### Carrying Out the Extension of Immobilized Primer Oligonucleotides with Fluorescently Labeled Nucleotides

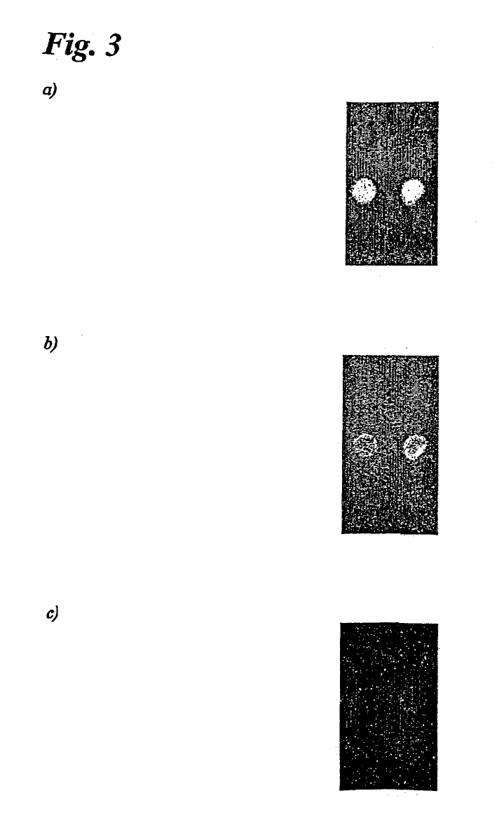

In the first step, a genomic sequence is treated with the use of bisulfite (hydrogen sulfite, disulfite) such that all of the cytosines not methylated at the 5-position of the base are modified such that a base that is different in its base-pairing behavior is formed, while the cytosines that are methylated in the 5-position remain unchanged. If bisulfite is used for the reaction, then an addition occurs at the unmethylated cytosine bases. In addition, a denaturing reagent or solvent as well as a radical trap must be present. A subsequent alkaline hydrolysis then leads to the conversion of unmethylated cytosine nucleobases to uracil. This DNA conversion serves for the purpose of detecting methylated cytosines. In the second step of the method, the treated DNA sample is diluted with water or an aqueous solution. A desulfonation of the DNA is then preferably conducted. In the third step of the method, the DNA sample is amplified in a polymerase chain reaction, preferably with a heat-stable DNA polymerase. In the present case, cytosines of the DAPK1 gene are investigated. For this purpose, a defined fragment with a length of 465 bp is amplified with the specific primer oligonucleotides ATTAATATTATGTAAAGTGA (SEQ-ID:1) and CTTACAACCATTCACCCACA (SEQ-ID:2). This amplified product serves as the template, which in turn serves for extending the immobilized primer oligonucleotides. After hybridization of the template to the immobilized primer oligonucleotides, these are extended in an extension reaction with the use of a nucleotide mixture of deoxynucleotides (here: dCTP, dTTP, dATP) and cyanine-5 (Cy5) or cyanine-3 (Cy3)-labeled deoxynucleotides (here: Cy5-dCTP, Cy3-dUTP). In FIG. 3a) the primer oligonucleotides are detected after hybridization with a Cy5-fluorescently-labeled amplified product of the DAPK1 gene and subsequent extension reaction with Cy3- and Cy5-fluorescently-labeled nucleotides, at a wavelength of 532 nm which is specific for the fluorescent dye Cy3. FIG. 3b) shows the signals detected via the fluorescently-labeled nucleotides incorporated in the extension reaction after subsequent dehybridization of the Cy5-fluorescently-labeled amplified product, at a wavelength of 532 nm. FIG. 3c) serves as the control; here, no signals can be detected at a wavelength of 532 nm after hybridization with the Cy5-fluorescently-labeled amplified product.

### EXAMPLE 2

### Carrying Out the Extension of Immobilized Primer Oligonucleotides with Nucleotides Modified with 5′-phosphothioate

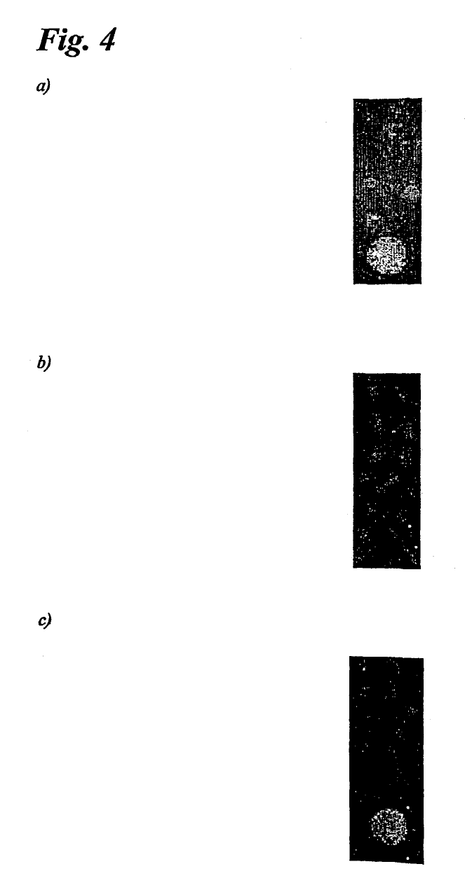

In the first step, a genomic sequence is treated with the use of bisulfite (hydrogen sulfite, disulfite) such that all of the cytosines not methylated at the 5-position of the base are modified such that a base that is different in its base-pairing behavior is formed, while the cytosines that are methylated in the 5-position remain unchanged. If bisulfite is used for the reaction, then an addition occurs at the unmethylated cytosine bases. In addition, a denaturing reagent or solvent as well as a radical trap must be present. A subsequent alkaline hydrolysis then leads to the conversion of unmethylated cytosine nucleobases to uracil. This DNA conversion serves for the purpose of detecting methylated cytosines. In the second step of the method, the treated DNA sample is diluted with water or an aqueous solution. A desulfonation of the DNA is then preferably conducted. In the third step of the method, the DNA sample is amplified in a polymerase chain reaction, preferably with a heat-stable DNA polymerase. In the present case, cytosines of the DAPK1 gene are investigated. For this purpose, a defined fragment with a length of 465 bp is amplified with the specific primer oligonucleotides ATTAATATTATGTAAAGTGA (SEQ-ID:1) and CTTACAACCATTCACCCACA (SEQ-ID:2). This amplified product serves as a template, which in turn serves for extending the immobilized primer oligonucleotides. After hybridization of the template to the immobilized primer oligonucleotides, the latter are extended in an extension reaction with the use of a nucleotide mixture of deoxynucleotides (here: dCTP, dTTP, dATP) and deoxynucleotides modified with 5′-phosphothioate (here: (α-S-dCTP, (α-S-dUTP). In FIG. 4a) the primer oligonucleotides are detected after hybridization with a Cy5-fluorescently-labeled amplified product of the DAPK1 gene and subsequent extension reaction with nucleotides modified with 5′-phosphothioate at a wavelength of 635* nm, which is specific for the fluorescent dye Cy5 FIG. 4b) shows the signals detected via the fluorescently-labeled nucleotides incorporated in the extension reaction after subsequent dehybridization of the Cy5-fluorescently-labeled amplified product, at a wavelength of 653 nm. In a subsequent step, all primer oligonucleotides which are not protected by the incorporation of a 5′-phosphothioate-modified nucleotide are hydrolyzed by addition of the enzyme phosphodiesterase 1 (PDE 1), which extensively hydrolyzes DNA from the 3′ terminus. In FIG. 4c) for primer oligonucleotides, which were protected by the incorporation of a 5′-phosphothioate-modified nucleotide prior to the hydrolysis of the PDE 1 enzyme, signals can be detected at a wavelength of 653 nm after hybridization with the Cy5-fluorescently-labeled amplified product of the DAPK1 gene. sic; 653?—Trans. Note.

1. A method for the highly parallel characterization of polymorphisms, hereby characterized in that the following steps are conducted:
a. a set of probes is bound to an addressed surface,
b. a nucleic acid to be investigated is hybridized to these probes;
c. the probes are extended in an allele-specific reaction, which depends on the sequence of nucleic acids to be investigated that functions as the template;
d. the probes are treated with a nuclease, which decomposes the unextended probes, but not the extended probes;
e. the remaining allele-specific extension products are analyzed.

2. The method according to claim 1, further characterized in that the address of the surface in step a) is the position (in an oligonucleotide array), a color, a fluorescent label, an isotopic label, a chemical label or a radioactive label.

3. The method according to one of the preceding claims, further characterized in that the nucleic acid to be investigated in step b) is genomic DNA, DNA pretreated with a bisulfite solution, cloned DNA, cDNA, RNA, a PCR product or a ligation product.

4. The method according to one of the preceding claims, further characterized in that the probes are converted to specific products, corresponding to step c), as a function of the respective sequence of the template hybridized thereon, by means of a polymerase and modified nucleotide building blocks.

5. The method according to one of claims 1 to 4, further characterized in that the probes are converted to specific extension products corresponding to step c), as a function of the respective sequence of the template hybridized thereon, by means of a ligase and a phosphorylated oligonucleotide.

6. The method according to one of the preceding claims, further characterized in that the extension reaction or the type of extension reaction depends on an SNP (Single Nucleotide Polymorphism) in the sample DNA.

7. The method according to one of the preceding claims, further characterized in that the nucleic acid to be investigated is a genomic DNA sample pretreated with a bisulfite solution (=hydrogen sulfite, disulfite) and that the extension reaction or the type of extension reaction depends on the methylation state of cytosine bases in the genomic DNA sample.

8. The method according to one of the preceding claims, further characterized in that SNPs and DNA methylation are investigated simultaneously.

9. The method according to one of the preceding claims, further characterized in that at least one nucleotide is attached in the extension reaction, which cannot be cleaved by a 3′-exonuclease or can be cleaved only with considerably reduced efficiency.

10. The method according to claim 1 or 9, further characterized in that this nucleotide is a methyl phosphonate, a phosphorothioate, a phosphorodithioate, a methyl phosphorothioate, an alkylated phosphorothioate or phosphorodithioate or a derivative of these compounds.

11. The method according to one of the preceding claims, further characterized in that a substituent which hinders decomposition by a 3′-exonuclease is attached to the concerned nucleotide base either on the nucleobase itself or on the deoxyribose.

12. The method according to one of the preceding claims, further characterized in that a 3′-exonuclease is used in step d).

13. The method according to claim 12, further characterized in that phosphodiesterase from Crotalus durissus (snake venom phosphodiesterase), Escherichia coli polymerase I, II, or III, T4 DNA polymerase, T7 DNA polymerase (unmodified), phosphodiesterase II type I-SA or calf thymus 54-kDa polypeptide with 3′-exonuclease activity is used as the 3′-exonuclease.

14. The method according to one of the preceding claims, further characterized in that the extension products are provided with a detectable label for detection.

15. The method according to one of claims 1 to 13, further characterized in that complementary oligomers are hybridized to the extension products, which are provided with a detectable label for detection.

16. The method according to claim 15, further characterized in that the complementary oligomers are oligonucleotides, RNA oligomers or PNA oligomers (Peptide Nucleic Acids).

17. The method according to one of claims 14 to 16, further characterized in that the labels are fluorescent labels.

18. The method according to one of claims 14 to 16, further characterized in that the labels are radionuclides.

19. The method according to one of claims 14 to 16, further characterized in that the labels are detachable mass labels, which are detected in a mass spectrometer.

20. The method according to one of claims 14 to 16, further characterized in that the extension products or the complementary oligomers themselves are detected by their mass in a mass spectrometer.

21. The method according to one of claims 1 to 14, further characterized in that the allele-specific extension products are analyzed by means of mass spectrometry.

22. The method according to one of claims 1 to 14, further characterized in that fragments of allele-specific extension products are analyzed by means of mass spectrometry.

23. The method according to one of claims 19 to 22, further characterized in that matrix-assisted laser desorption/ionization mass spectrometry (MALDI) or electrospray ionization mass spectrometry (ESI) is used for analysis

24. The method according to claim 15 or 16, further characterized in that the probes or the complementary oligomers are present in a form which is particularly well suitable for mass-spectrometric analysis.

25. The method according to claim 24, further characterized in that particularly good suitability for mass-spectrometric analysis is achieved if the allele-specific products have a net single positive charge or single negative charge.

26. The method according to one of the preceding claims, further characterized in that a plurality of different probes are [present] on one addressed analysis point of the surface.

27. The method according to one of the preceding claims further characterized in that known polymorphisms in the DNA to be investigated are genotyped.

28. The method according to one of claims 1 to 26, further characterized in that unknown polymorphisms in the DNA to be investigated are identified.

27. The method according to one of the preceding claims, further characterized in that cytosine methylations are detected and visualized.

28. The method according to claim 27, further characterized in that known methylation patterns are investigated in the sample to be analyzed.

29. The method according to one of the preceding claims, wherein the genomic DNA is obtained from a DNA sample, whereby sources for DNA include, e.g., cell lines, blood, sputum, stool, urine, cerebrospinal fluid, tissue embedded in paraffin, for example, tissue from eyes, intestine, kidney, brain, heart, prostate, lungs, breast or liver, histological microscope slides and all possible combinations thereof.

30. Use of a method according to one of the preceding claims, for the diagnosis and/or prognosis of adverse events for patients or individuals, whereby these adverse events belong to at least one of the following categories: undesired drug interactions; cancer disorders; CNS malfunctions, damage or disease; symptoms of aggression or behavioral disturbances; clinical, psychological and social consequences of brain damage; psychotic disturbances and personality disorders; dementia and/or associated syndromes; cardiovascular disease, malfunction and damage; malfunction, damage or disease of the gastrointestinal tract; malfunction, damage or disease of the respiratory system; lesion, inflammation, infection, immunity and/or convalescence; malfunction, damage or disease of the body as an abnormality in the development process; malfunction, damage or disorder of the skin, the muscles, the connective tissue or the bones; endocrine and metabolic malfunction damage or disorder headaches or sexual malfunction.

31. Use of a method according to one of the preceding claims for distinguishing cell types or tissues or for investigating cell differentiation.

32. A kit containing at least one primer pair for amplification, a set of probes and enzymes and buffer and instructions for conducting the method according to one of claims 1 to 29.
